(19) United States
(12) Plant Patent Application Publication
(10) Pub. No.: US 2010/0293686 P1
(43) Pub. Date: Nov. 18, 2010

(54) Alstroemeria plant named 'Tangerine Tango'

(50) ;

(75) Inventor: Mark P. Bridgen, Southold, NY (US)

Correspondence Address: JONDLE & ASSOCIATES P.C., 858 HAPPY CANYON ROAD SUITE 230, CASTLE ROCK, CO, 80108, US

(73) Assignee: Cornell University, Ithaca, NY (US)

(21) Appl. No.: 12/800,202

(22) Filed: May 11, 2010

Related U.S. Application Data
(60) Provisional application No. 61/216,010, filed on May 12, 2009.

(51) Int. Cl. A01H 5/00

(52) U.S. Cl. PLT/309

(57) ABSTRACT

An Alstroemeria variety particularly distinguished by bright orange inflorescences with yellow highlights, good longevity as a cut flower, continuous flowering from summer through fall, and winter-hardy from USDA zone 5 or warmer, is disclosed.

### CROSS REFERENCE TO RELATED APPLICATIONS

This application claims priority under 35 U.S.C. §119 from U.S. Provisional Application No. 61/216,010, filed on May 12, 2009, which is incorporated herein in its entirety by reference.

### ACKNOWLEDGEMENT OF FEDERAL RESEARCH SUPPORT

This invention was made, at least in part, with government support under Hatch Funding from the U.S. Department of Agriculture, Project Number NYC-145302, in cooperation with Cornell University's State Agricultural Experiment Stations. Accordingly, the United States government has certain rights in this invention.

### GENUS AND SPECIES

Alstroemeria hybrid L.

### VARIETY DENOMINATION

‘Tangerine Tango’

### BACKGROUND OF THE NEW PLANT

The present invention comprises a new and distinct variety of Alstroemeria, botanically known as Alstroemeria hybrid L., and hereinafter referred to by the variety name ‘Tangerine Tango’. ‘Tangerine Tango’ originated from a hybridization made in April 2002 in Riverhead, N.Y. The female parent was an un-named individual plant of Alstroemeria aurea plant (unpatented), while the male parent was a proprietary Alstroemeria plant ‘00-0203’ (unpatented).

‘Tangerine Tango’ was selected for its distinctive bright orange inflorescences with yellow highlights, continuous flowering and strong, upright flower stems.

‘Tangerine Tango’ has been asexually propagated repeatedly by in-vitro micropropagation and whole-plant division of rhizomes in Riverhead, N.Y. since Fall 2002 for approximately ten generations and has been found to retain its distinctive characteristics through successive asexual propagations.

### SUMMARY OF THE INVENTION

The following are the most outstanding and distinguishing characteristics of this new variety when grown under normal commercial practices in a greenhouse or outdoor field in Riverhead, N.Y.

1. Bright orange inflorescences with yellow highlights;

2. Has good longevity as a cut flower;

3. Continuous flowering from summer through fall; and

4. Winter-hardy from USDA zone 5 or warmer.

### DESCRIPTION OF THE PHOTOGRAPHS

This new Alstroemeria plant is illustrated by the accompanying photographs which show blooms and foliage of the plant. The colors shown are as true as can be reasonably obtained by conventional photographic procedures. The plants in the photographs are approximately one year old.

FIG. 1 shows the overall plant habit, including mature inflorescences, flower buds, and leaves.

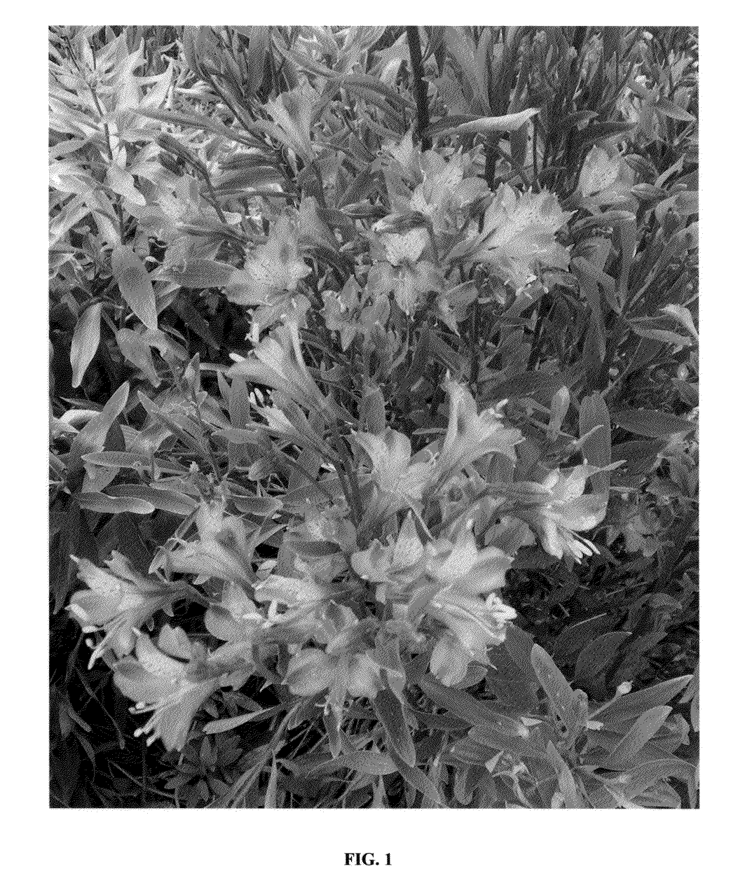

FIG. 2 shows a close-up of the mature inflorescence.

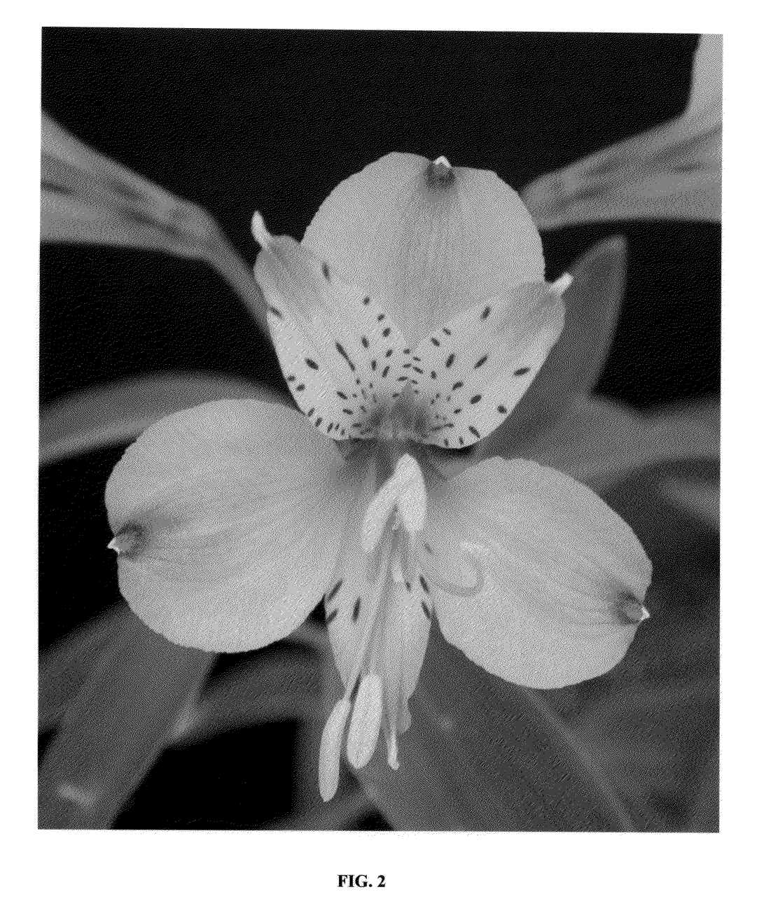

### DESCRIPTION OF THE NEW VARIETY

The following detailed descriptions set forth the distinctive characteristics of ‘Tangerine Tango’. The data which define these characteristics were collected from asexual reproductions carried out in Riverhead, N.Y. from approximately one-year-old plants. The data were collected in the summer and fall on plants grown under both field conditions and in a greenhouse under natural light. Color references are primarily to the R.H.S. Colour Chart of The Royal Horticultural Society of London (R.H.S.), 2001.

### DETAILED BOTANICAL DESCRIPTION

Classification:
   Botanical.—Alstroemeria hybrid L.
   Common name.—Alstroemeria, Lily-of-the-Incas, Inca Lily, Peruvian Lily.
   Denomination.—‘Tangerine Tango’.
Parentage:
   Female parent.—An un-named individual plant of Alstroemeria aurea plant (unpatented).
   Male parent.—The proprietary Alstroemeria plant ‘00-0203’ (unpatented).
Plant:
   Form.—Herbaceous plant arising from an underground rhizome.
   Habit.—Upright and vigorous.
   Plant height.—75.0 cm to 80.0 cm.
   Plant width (spread).—90.0 cm to 100.0 cm.
   Flowering habit.—Continuous flowering from the beginning of summer until the first hard freeze in fall.
   Time to produce a finished flowering plant.—If a clump is divided in the spring or early summer, it will resume flowering within a month.
Flowering stems:
   Color.—RHS 137A or RHS 137C.
   General description.—Long, strong, and upright growing floral stems.
   Stem strength.—Strong.
   Length.—62.5 cm to 80.0 cm.
   Diameter.—1.0 cm to 2.0 cm.
   Internode length.—Not applicable.
Leaves:
   Arrangement.—Simple, linear, resupinate leaves with parallel venation that is arranged alternately.
   Shape.—Elongated and elliptical with extended bases.
   Apex.—Broadly acuminate.
   Base.—Extended.
   Margin.—Entire.
   Color, mature leaf (both surfaces).—RHS 137A or RHS 137C.
   Length.—The vegetative stem is 10.0 cm.
   Width.—The vegetative stem is 2.2 cm.
   Texture.—Glabrous.
   Venation pattern.—Parallel.
   Pubescence.—Absent.
   Petiole.—Absent (sessile leaves).
Inflorescence:
   Type.—A terminal bracted-umbel composed of cymes.
   Flowering season.—Early summer through early fall.
   Lastingness of inflorescences on the plant.—15 to 21 days.
   Color.—RHS 70B to RHS 71B.
   Flower width.—Approximately 5.0 cm at the open end.
   Flower depth.—3.7 cm to 4.2 cm.
   Flower shape.—Tubular.
   Fragrance.—Absent.
   Quantity of individual flowers per plant.—Everblooming; varies with size of plant; 30 to 50 every three months.
   Number of florets per umbel.—Typical: 15 Range: 10 to 20.
   Peduncle.—Length: 2.5 cm to 4.0 cm Diameter: Approximately 0.9 cm to 1.0 cm before opening.
Flower buds:
   Color.—RHS 70B to RHS 71B.
   Shape.—Pear-shaped and becoming long and more pointed just before opening.
   Length.—Approximately 2.5 cm to 2.8 cm before opening.
   Diameter.—Approximately 0.9 cm to 1.0 cm before opening.
Tepals:
   Arrangement.—Composed of two concentric circles of three tepals each.
   Shape.—Tubular/spatulate to oblanceolate-spatulate.
   Apex.—Emarginate to mucronate to apiculate.
   Length.—3.7 cm to 4.2 cm.
   Width.—Approximately 5.0 cm at the tip.
   Texture.—Glabrous.
   Color.—RHS 71B on young flowers and fading slightly to RHS 70B on older flowers.
Reproductive organs:
   Pistils.—Quantity: 1 Style length: 2.5 cm to 4.0 cm.
   Stamens.—Quantity: 6 Filaments: Length: Approximately 2.5 cm to 4.0 cm Pollen: Flowers are sterile; no pollen is produced.
Seeds: No seeds have been observed.
Disease and insect resistance: Because this plant is sterile and no pollen is produced, thrips are less likely to attack. Disease resistance is typical of the species.

### COMPARISON WITH PARENTAL AND COMMERCIAL VARIETIES

‘Tangerine Tango’ differs from the female parent, an un-named individual plant of Alstroemeria aurea (unpatented), in that ‘Tangerine Tango’ has larger inflorescences and shorter peduncles than the female parent. Additionally, ‘Tangerine Tango’ is everblooming and blooms for a longer period of time than the female parent.

‘Tangerine Tango’ differs from the male parent, the proprietary Alstroemeria plant ‘00-0203’ (unpatented), in that ‘Tangerine Tango’ has larger inflorescences and a longer blooming time than ‘00-0203’.

‘Tangerine Tango’ differs from the commercial Alstroemeria variety ‘Mauve Majesty’ (U.S. Plant Pat. No. 18,183), by having orange inflorescences with yellow highlights, while ‘Mauve Majesty’ has pale-purple inflorescences.

I claim:

1. A new and distinct variety of Alstroemeria plant named ‘Tangerine Tango’ as described and shown herein.
